(19) United States
(12) Patent Application Publication
(10) Pub. No.: US 2014/0336648 A1
(43) Pub. Date: Nov. 13, 2014

(54) ADJUSTABLE DYNAMIC EXTERNAL FIXATOR

(71) Applicant: HOPITAUX UNIVERSITAIRES DE GENEVE, Geneve (CH)

(72) Inventors: Jan Van Aaken, Geneve (CH); Kun Liu, Beijing (CN)

(73) Assignee: HOPITAUX UNIVERSITAIRES DE GENEVE, Geneve (CH)

(21) Appl. No.: 14/275,926

(22) Filed: May 13, 2014

(30) Foreign Application Priority Data
May 13, 2013 (EP) 13167518.3

(51) Int. Cl. A61B 17/66

(52) U.S. Cl. CPC A61B 17/66; A61B 2017/606
USPC 606/58

(57) ABSTRACT

Dynamic external fixator comprising a pair of rods (1) made of a radiolucent material, each rod (1) comprising a base (10), preferably with an opening (13), a stem (11) extending from the base (10), with a sliding groove (14) extending along a longitudinal axis of the stem (11); and stopping means (12, 2) between the base (10) and the stem (11); a pair of springs (3), each spring (3) being arranged on the stem (11) of one of the rods (1) and abutting against the stopping means (12, 2); proximal attachment means (K1) for attaching the base (10) of each rod (1) to a first position; distal attachment means (K2) for attaching the stem (11) of each rod (1) to a second position, wherein the distal attachment means (K2) are inserted into the spirals of the spring (3) and through the sliding groove (14) of each of the rods (1), such that turning the spring (3) around the stem (11) allows adjusting a distraction force between the proximal attachment means (K1) and the distal attachment means (K2); and method for attaching such a dynamic external fixator.

The present invention relates to an adjustable dynamic external fixator for fracture dislocations and/or for flexion contracture of joint. The present invention relates in particular to an adjustable dynamic external fixator for the distraction of fracture dislocations of the proximal interphalangeal (PIP) joint and/or for distraction of the elbow or knee joint, for example in case of joint stiffness.

Unstable fracture dislocations of the proximal interphalangeal (PIP) joint present challenges for hand surgeons as they frequently lead to persistent pain, stiffness, instability and posttraumatic degenerative arthritis. They have an estimated incidence of 9 fractures per 100,000 persons per year.

The stability of the PIP joint is determined by the size of the volar fragment of the base of the middle phalanx and the degree of impaction of the remaining dorsal articular surface. If the volar fragment is larger than 40% of the joint surface as seen on a lateral radiograph, it is likely that all or most of the collateral ligaments are attached to it, thus leading to dorsal instability of the middle phalanx.

In the treatment of these injuries, only large fragments are amendable to open reduction and internal fixation (ORIF). This is not possible in comminuted fractures which represent the majority of the cases. The management of comminuted fractures includes immobilization in substantial flexion of the PIP joint, extension block splinting, or extension block pinning, transarticular wiring, closed reduction and percutaneous pins, ORIF with pin fixation, volar plate arthroplasty, hemi-hamate arthroplasty, volar plating and hemi-arthroplasty.

An alternative technique, consisting in static traction with rubber bands, has first been published as early as 1946 by Robertson, R. C.; Cawley, J. J., Jr.; and Faris, A. M. in “Treatment of fracture-dislocation of the interphalangeal joints of the hand.” J Bone Joint Sung Am. 1946; 28: 68-70. Three decades later Salter, R. B.; Simmonds, D. F.; Malcolm, B. W.; Rumble, E. J.; MacMichael, D.; and Clements, N. D. in “The biological effect of continuous passive motion on the healing of full-thickness defects in articular cartilage. An experimental investigation in the rabbit.”, J Bone Joint Surg Am. 1980; 62(8): 1232-51., demonstrated that joint movement enhances cartilage regeneration and healing.

According to these findings R. R. Schenck in “Dynamic traction and early passive movement for fractures of the proximal interphalangeal joint.”, J Hand Surg Am. 1986; 11(6): 850-8., described the principle of dynamic traction, the combined use of traction and movement, and applied it to the PIP joint by designing a dynamic external fixator. He postulated that traction on the bony fragments by ligament and volar plate attachments (ligamentotaxis) would lead to reduction of articular fragments. Secondly, he stated that maintenance of traction until consolidation of the fracture would avoid collapse of the fragments. And furthermore he believed that distraction avoided contracture of the joint ligaments and other periarticular structures, a major contributor to joint stiffness. Active or passive movement allows for tendon gliding and diminishes the formation of intraarticular and periarticular adhesions.

Many dynamic external fixators have been designed since then. However, all of these devices that can be used to treat unstable injury of the PIP joint have one or more drawbacks. Some devices obscure the fracture site on true lateral radiograms with non-radiotransparent material, like K-wires, force couple splint or springs. Some are cumbersome; some are time-consuming as they need to be assembled during the operation; some don't allow for differential traction on either side; some do not allow for adjustment of the distraction force during the traction period; and others are rigidly hinged which have an unacceptable high rate of complications.

There is thus a need for a dynamic external fixator that allows overcoming the shortcoming of the above fixators.

An aim of the present invention is to provide a dynamic external fixator that doesn't obscure the fracture site on a true lateral view with fluoroscopy or standard radiographs.

Another aim of the invention is to provide a dynamic external fixator that is ready to use and easy to install.

Still another aim of the invention is to provide a dynamic external fixator that is not bulky and adjustable, where distraction can preferably be altered independently on either side during distraction period.

These aims and other advantages are achieved with the dynamic external fixator and with a method for attaching a dynamic external fixator according to the corresponding independent claim.

These aims and other advantages are achieved in particular with a dynamic external fixator comprising a pair of rods made of a radiolucent material, each rod comprising a base, a stem extending from the base, with a sliding groove extending along a longitudinal axis of the stem; and stopping means between the base and the stern; a pair of springs, each spring being arranged on the stem of one of the rods and abutting against the stopping means; proximal attachment means for attaching the base of each rod to a first position; distal attachment means for attaching the stem of each rod to a second position, wherein the distal attachment means are inserted into the spirals of the spring and through the sliding groove of each of the rods, such that turning the spring around the stem allows adjusting a distraction force between the proximal attachment means and the distal attachment means.

In embodiments, the base comprises an opening for receiving the proximal attachment means.

In embodiments, the stopping means comprises a shoulder formed between the base and the stem, or a spacer made of radiolucent material placed on the stem and abutting against a shoulder of the base. The stem for example comprises a screw thread matching a screw thread of the spacer, wherein the position of the spacer on the stem is determined by bolting/unbolting the spacer on the stem.

In embodiments, the stopping means comprises a plurality of spacers made of radiolucent material and placed next to each other on the stem.

The proximal and/or distal attachment means are for example K-wires, Steinmann wires, or any other appropriate attachment means.

In variant embodiments, the dynamic external fixator further comprises reduction attachment means for attaching the stem of each rod to a reduction position between the proximal position and the distal position, wherein the reduction attachment means are inserted into the spirals of the spring and through the sliding groove of each rod, such that the reduction attachment means can be moved within said sliding groove by turning the spring around the stem. The reduction attachment means is for example a K-wire, a Steinmann wire, or any other appropriate attachment means.

These aims and other advantages are also achieved with a method for attaching such a dynamic external fixator, comprising the steps of attaching the proximal attachment means to a first position, attaching the distal attachment means to a second position, selecting a pair of rods with appropriate position of the stopping means, placing a spring on the stem of each rod, attaching the proximal attachment means to the base of each rod; inserting the distal attachment means through the spirals of each spring and through the sliding groove of each rod; adjusting for each rod the distraction force between the proximal attachment means and the distal attachment means by turning the corresponding spring.

In embodiments, the method further comprises the step of attaching reduction attachment means to a third position between the first and the second position and inserting the reduction attachment means through the spirals of each spring and through the sliding groove of each rod.

The step of selecting a pair of rods with appropriate position of the stopping means for example comprises selecting a pair of rods with appropriate dimensions of the base along a longitudinal axis of said rod and/or selecting one or more spacers for placement on the stem of each rod in abutment against the base of the corresponding rod and/or adjusting a distance between the base and a spacer by bolting/unbolting the spacer on the stem of the corresponding rod.

The fixator of the invention and the corresponding attaching method provide for a simple construction and attachment procedure; for an easy regulation of the traction exerted by the fixator; and for flexibility in adjusting the distance between the first attachment means and the springs for creating a suitable space in which an X-ray image will not be obscured.

The present invention will be better understood with the help of the following description of preferred embodiments, illustrated by the figures where:

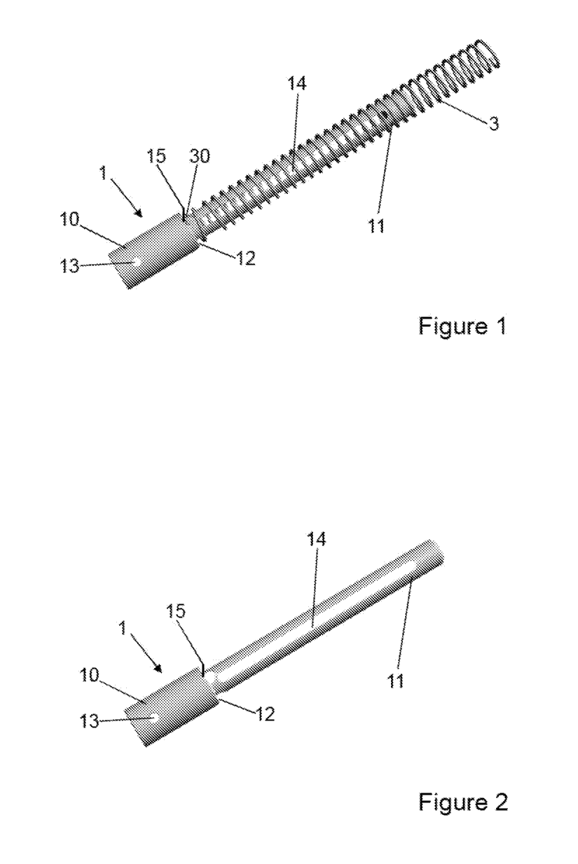

FIG. 1 illustrates a part of an assembled dynamic external fixator according to an embodiment of the invention.

FIG. 2 shows a rod of the dynamic external fixator partially illustrated in FIG. 1.

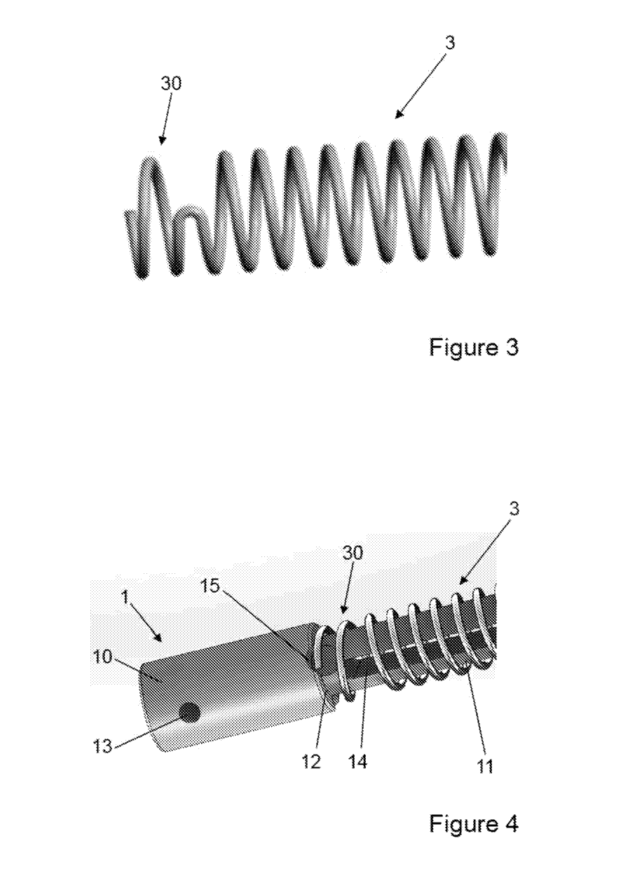

FIG. 3 shows a proximal end of a spring of the dynamic external fixator partially illustrated in FIG. 1.

FIG. 4 is a detail of the base of a rod of the dynamic external fixator partially illustrated in FIG. 1.

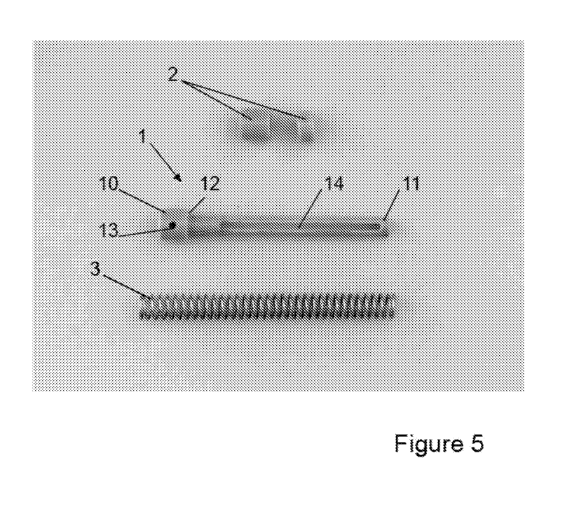

FIG. 5 shows parts of a disassembled dynamic external fixator according to a variant embodiment of the invention.

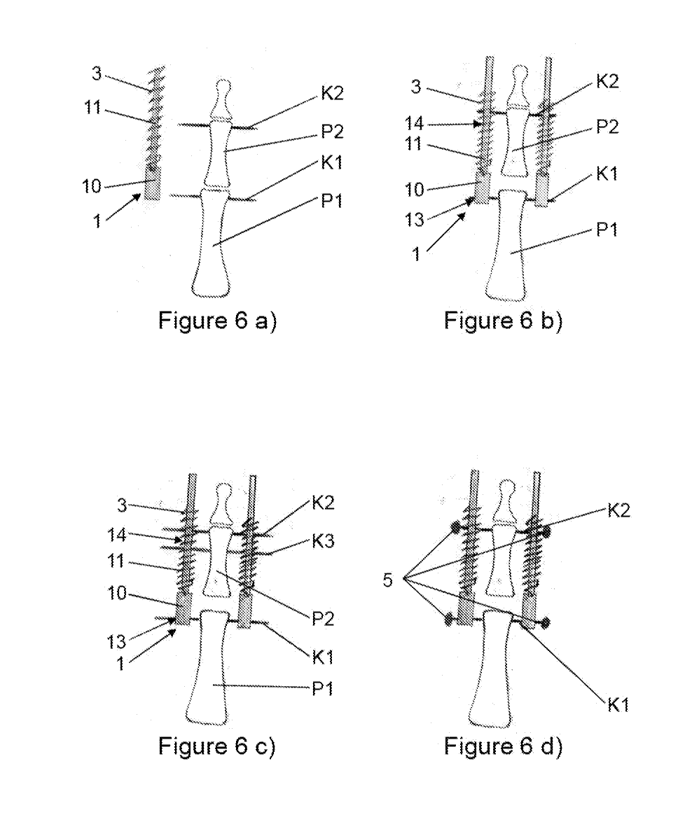

FIGS. 6a) to d) show dynamic external fixators according to embodiments of the invention at various steps of their application to a finger.

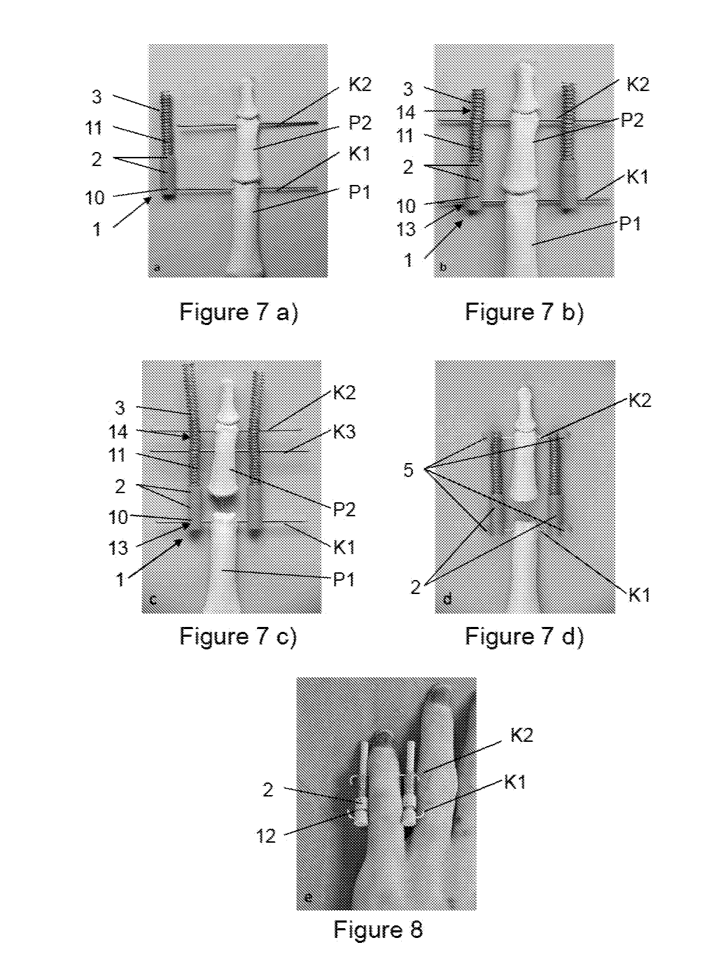

FIGS. 7a) to d) show dynamic external fixators according to variant embodiments of the invention at various steps of their application to a finger.

FIG. 8 shows a dynamic external fixator according to a variant embodiment of the invention affixed to a fractured finger, as viewed form above.

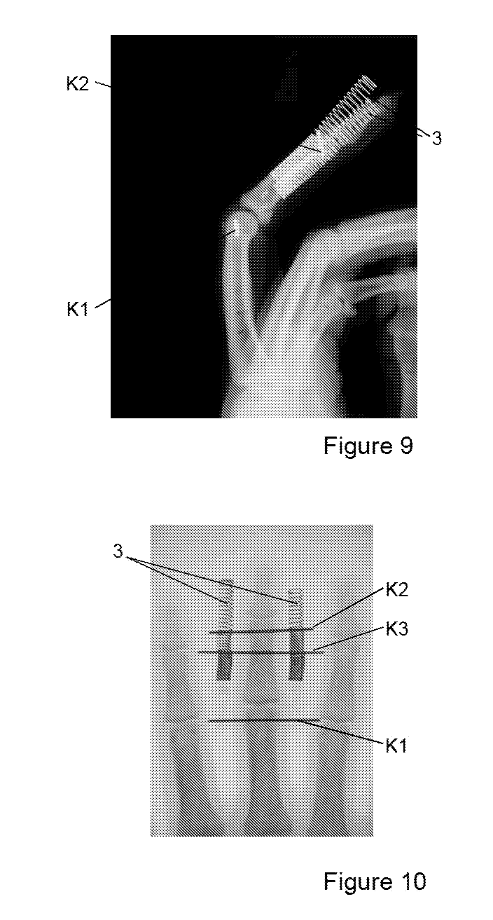

FIG. 9 is a lateral X-ray image of a fractured PIP joint distracted by a dynamic external fixator of the invention.

FIG. 10 is an antero-posterior X-ray image of a fractured PIP joint distracted by a dynamic external fixator according to a variant embodiment of the invention.

FIG. 1 shows elements of the dynamic external fixator according to embodiments of the invention. Accordingly, the fixator comprises a pair of rods 1, of which only one is visible in FIG. 1, and one spring 3 per rod 1. The rods 1 are made of a solid radiolucent material such as for example Polyether Ether Ketone (PEEK) and/or fibrocarbon, and/or any other appropriate solid radiolucent material. The springs 3 are for example made of metal, for example of stainless steel, or any appropriate material.

With reference to FIGS. 1 and 2, each rod 1 comprises a base 10 and a stem 11 extending from said base 10 along a longitudinal axis of the rod 1. The base 10 is for example cylindrical and aligned along the same longitudinal axis. Other shapes of the base 10, in particular sections of the base 10 other than circular, are however possible within the frame of the invention. The outer dimensions of the base 10, for example its diameter, are preferably larger than the outer dimensions, for example the diameter, of the stem 11. The base 10 thus forms a shoulder 12 at a proximal end of the stem 11.

The base 10 comprises an opening 13, for example a rotational hole, for receiving attachment means for attaching the proximal end of the rod 1 to a bone of a patient, for example to a proximal phalanx. The rotational hole 13 is for example provided in the center of the base 10 and oriented perpendicular to the rod's 1 longitudinal axis. The rotational hole 13 for example has a diameter of 1.4 mm, and allows the insertion of a K-wire through the base 10, as will be explained in more details below.

The stem 11 comprises a sliding groove 14 that extends along at least part of its length for receiving attachment means for adjustably attaching the distal end of the rod 1 to a bone of a patient, for example to an intermediate phalanx. In embodiments, the sliding groove 14 extends in a plane defined by the opening 13 and the rod's 1 longitudinal axis. In embodiments, the groove 14 has a length of 30 mm and a width of 1.4 mm, and allows the insertion of one or more K-wires through the stem 11. Other dimensions of the groove are however possible within the frame of the invention, depending for example on the intended use of the fixator and/or the size of the K-wires.

In embodiments, the length of the base 10 along the rod's longitudinal axis is determined for creating a window of a desired dimension between the opening 13 and the shoulder 12 that is free of any material that may obscure an X-ray image, for example a lateral X-ray image of the fracture. In embodiments, there are different rods 1 with bases of different sizes for the practitioner to choose from in order to form a fixator with window dimensions appropriate for the intended use or positioning around the fracture.

With reference to FIGS. 1 and 2, the spring 3 is dimensioned to be slidably engageable with the stem 11 of the rod 1 and to abut against the shoulder 12. The spring 3 is for example a stainless steel spring having a length of 5 cm, and an inside diameter of 4 mm, a coil distance of 1.5 mm and a wire diameter of 0.5 mm. Other dimensions are of course possible within the frame of the invention, depending in particular on the dimensions of the rod 1, and/or on the intended use of the fixator.

In embodiments, and with reference to FIGS. 1 to 4, the base 10 comprises retaining means 15, for example in the form of a groove formed radially in the shoulder 12, for preventing the spring 3 to unwantedly turn around the rod 1 at least in one direction. The retaining means for example prevents unwanted rotation of the spring 3 in a direction that would release the pressure on the spring 3 when the fixator is installed, thereby preventing un unwanted reduction of the traction effect of the fixator on the fractured finger once installed.

In embodiments, the retaining means 15 acts on the proximal end 30 of the spring 3 to maintain it in a desired position relative to the rod 1 when the spring 3 is in contact with the shoulder 12. The proximal end 30 for example has a pitch that is inverted relative to the rest of the spring 3 on one to two turns in order to form a hook that enters the retaining means 15 when the spring 3 abuts the shoulder 10 in a particular radial arrangement.

With reference to FIG. 2, the retaining means 15 is for example formed by drilling a hole in the rod 1, radially to the longitudinal axis of the rod 1 and at the junction between the base 10 and the stem 11, thereby creating a radial groove in the shoulder 12, possibly two groves separated by 180° around the rod's 1 longitudinal axis.

The retaining means 15 and the proximal end 30 of the spring 3 are for example configured such that the spring 3 can be freely turned in one direction around the rod 3 when the spring 3 abuts against the shoulder 12, but is prevented by the retaining means 15 to turn more than one turn in the opposite direction. In order to turn the spring 3 in the opposite direction, for example in order to reduce the traction effect of the installed fixator, the spring 3 must be disengaged from the retaining means 15, for example by pushing the proximal end 30 of the spring 3 away from the base 10 and thus of the shoulder 12, before being turned.

In a variant embodiment illustrated in FIG. 5, the fixator further comprises one or more spacers 2 of possibly different sizes in order to adjust the distance between the proximal end of the spring 3 and the radial opening 13. The one of more spacers 2 are made of a solid radiolucent material such as for example Polyether Ether Ketone (PEEK) and/or fibrocarbon, and/or any other appropriate solid radiolucent material, similar or identical to the material of the rods 1. Adjusting the distance between the proximal end of the spring 3 and the radial opening 13 leads to adjusting the size of a window free of any material that may obscure an X-ray image, for example a lateral X-ray image of the fracture.

The spacer 2 is configured for being slideably engageable around the stem 11 of the rod 1. The spacer 2 for example comprises a central opening allowing insertion of the stem 11 there through. The central opening is dimensioned such that the spacer 2 will abut against the shoulder 12 when sliding along the stem 11, i.e. the dimensions of the spacer's central opening, for example its diameter, are smaller than the base's external dimensions, for example its external diameter. In embodiments, the spacer 2 further comprises a lateral opening perpendicular to its central opening for the insertion of additional attachment means there through. The lateral opening for example has a diameter of 1.2 mm, which allows the insertion of a 1.2 mm K-wire therein. The lateral opening could then be used as a drill guide when inserting attachment means for attaching the fixator of the invention.

In embodiments, the stem 11 comprises screw threads on at least part of its proximal end, to fit screw threads formed within the central opening of the spacer 2. The distance between the spacer 2 and the base 10, in particular the distance between the opening 13 and the distal edge of the spacer 2, is for example adjusted by choosing spacers 2 of appropriate length and/or by placing several spacers along the stem 11, such that when the one or more spacer are bolted on the stem 11 against the base 10, the desired distance is achieved. Alternatively, the distance between the opening 13 and the distal edge of the spacer 2 can be adjusted by screwing or unscrewing the spacer 2 on the stem 11. For example, the dimensions of the screw thread on the stem 11 are 4.5×0.5 mm and its length is 8 mm. Other dimensions are of course possible within the frame of the invention, depending in particular on the dimensions of the stem 11.

In other embodiments, the spacer 2 can freely slide along the stem 11 until it abuts against the shoulder 12, i.e. the stem 11 and the spacer 2 do not comprise any screw thread. The distance between the base 10, in particular the opening 13 and the distal end of the spacer 2 is then adjusted by choosing a spacer 2 of corresponding length and/or by placing several spacers along the stem 11.

The length of the spacer 2, with or without screw thread in its central opening, is for example 6 mm, 8 mm, 10 mm, 12 mm, 14 mm, or any other appropriate length, depending on the need, for example on the intended position and use of the fixator of the invention.

According to the invention, the dynamic external fixator thus comprises a pair of rods 1, each rod 1 having a spring 3 slidably arranged on the stem 11, the appropriate distance between the opening 13 and the proximal end 30 of the spring 3 being determined by the length of the base 10, i.e. the distance between the opening 13 and the shoulder 12. In variant embodiments, one or more spacers 2 are arranged on the stem 11 between the base 10 and the spring 3 for determining an appropriate distance between the opening 13 and the proximal end of spring 3.

The dynamic external fixator further comprises attachment means for attaching the base 10 and the stem 11 of each rod 1, each to another part of a patients body, the bases 10 being attached for example on either sides of a proximal phalanx, while the stems 11 are attached on the respective sides of the intermediate phalanx. The attachment means are for example K-wires to be inserted in the openings 13 and in the sliding grooves 14. Other attachment means having other sizes as appropriate for the intended use or positioning of the fixator may be used within the frame of the invention.

FIGS. 6a) to 6d) schematically illustrate the installation of dynamic external fixators according to embodiments of the invention onto a human finger for applying dynamic traction to a fracture-dislocated PIP-joint.

In a first step illustrated in FIG. 6a), the attachment means are attached to the phalanxes P1, P2 on either side of the broken joint. The attachment means are for example two K-wires K1, K2. A proximal K-wire K1, for example with a diameter of 1.2 mm, is inserted through the rotational center of the head of proximal phalanx P1 and a distal K-wire K2 is inserted through the rotational center of the head of intermediate phalanx P2. The length of the base 10 of each rod 1 is determined in order to un-obscure the PIP-joint in true lateral X-ray views when installed onto the fractured finger.

In a next step, the rods 1 are then attached on either side of the finger as illustrated in FIG. 6b), with their base 10 attached to the proximal K-wire K1 and their stem 11 attached to the distal K-wire K2. The proximal K-wire K1 is for example inserted through the opening 13, while the distal K-wire K2 is inserted between the spirals of the springs 3 and through the sliding groove 14.

In a next step, the springs 3 are turned for example in clockwise direction for increasing the distraction of the fractured PIP joint, thereby reducing the fracture fragments and the PIP joint subluxation. The distraction is adjusted independently on each side of the fractured finger by turning each spring 3 independently.

In variant embodiments illustrated for example in FIG. 6c), the fixator comprises a third attachment means located between the proximal attachment means and the distal attachment means. For example, an optional reduction pin is inserted into the middle phalanx between the distal pin and the one or more spacers 2 through the sliding groove 14, to enhance the stability of the fixator. The third attachment means, or reduction pin, is for example a reduction K-wire K3 inserted into the middle phalanx, through the spirals of the spring 3 of each rod 1, and through the sliding groove 14 of each rod 1, between the distal K-wire K2 and the spacer 2, if any, or the shoulder 12. The reduction K-wire K3 can be beneficial, for example, if subluxation occurs during motion of the PIP joint under lateral fluoroscopy after applying the fixator of the invention. More generally, the reduction attachment means enhance the stability of the joint and fracture.

In an optional step illustrated in FIG. 6d), the excessive spirals are cut off at the end of the rods 1. The K-wires K1, K2 are bent for example at 90° to block the rods 1 and excessive length is cut, for example at 5 mm and pin balls 5 and/or other protection elements are optionally put on their extremities.

Optionally, rubber or plastic caps, or any other appropriate retaining means, are attached to, for example inserted or bolted on, the distal extremities of the stems 11 for preventing the springs 3 from falling off the rods 1.

FIGS. 7a) to 7d) illustrate the same steps as illustrated in FIGS. 6a) to 6d), but in the case of the installation of an external fixator comprising spacers 2 on the rods 1. The steps for installing the fixator according to this variant embodiment are similar to the ones described above in relation with the embodiment illustrated in FIGS. 6a) to 6d), except for the installation of the spacers 2 that is illustrated on FIG. 7a), wherein the spacers 2 are for example slid and/or bolted onto the stems 11 of the rods 1. The length and/or number of spacers 2 are chosen in order to create a suitable space in which the PIP-joint will not be obscured in true lateral X-ray views.

As visible in FIG. 8 the thus formed an installed dynamic external fixator is particularly compact, while achieving reduction of the fracture fragment displacement and PIP joint subluxation, as can be seen in FIG. 9.

The dynamic external fixator of the invention furthermore provides for a window between the proximal K-Wire K1 and the shoulder 12, or in embodiments between the proximal K-Wire K1 and the distal end of the spacer 2, that is free of any material that may obscure a lateral X-ray image of the fractured joint, as illustrated in FIG. 8. Indeed, the springs 3 installed on each rod 1 are maintained out of this window by the stopping means and the base 10 of each rod 1 and/or, in embodiments, the spacers 2, are made of a radiolucent material. Appropriately dimensioning the window thus allows a practitioner to take an X-Ray image of the fracture, in particular a lateral X-Ray image of the fracture, that will not be obscured by any part of the external fixator.

Experiment

Preparation of the Fracture Dislocation Model of PIP Joints

One long finger and one little finger in the same adult fresh-frozen hand specimen were used in this study. The specimen was evaluated radiographically to exclude any fractures, dislocations or other lesions. Through a palmar approach, the palmar plate was exposed and left untouched. A 1.0 mm K-wire was used to plan the osteotomy. The wire was directed to the midline of the articulation in the lateral view in order to make an osteotomy with osteotome and hammer that mobilized the palmar half of the articular surface of the middle phalanx. The digital flexor and extensor tendons of the long and little fingers were then pulled at wrist level thus creating a dorsal subluxation of the PIP joint under lateral fluoroscopy control. The incision was closed with sutures in standard fashion.

Applying the Dynamic External Fixator

Under fluoroscopy the dynamic external fixator was applied as explained above.

Under fluoroscopy screening in a lateral position, the PIP joint was moved passively by pulling on the digital flexor and extensor tendons at wrist level. The fluoroscopy images as well as standard radiographs were obtained to evaluate the congruency of the joint surfaces between the base of the middle phalanx and the head of the proximal phalanx.

The osteotomy mobilized approximately one-half of the palmar surface of the middle phalanx of the PIP joint. Dorsal subluxation of the middle phalanx occurred when the PIP joint was passively moved.

After applying the fixator the PIP joint surface could be clearly seen on a lateral view in fluoroscopy without any obstruction by the rods or nuts. Once distraction was exerted by the fixator of the invention, lateral fluoroscopy showed complete reduction of the fracture and correction of the subluxation of the PIP joint. The articular surface of the base of the middle phalanx appeared smooth and was congruent with the head of the proximal phalanx (FIG. 9). The PIP joint could be moved smoothly and reduction was maintained throughout full range-of-motion (ROM) with or without a reduction K-wire K3. The traction force could be easily increased or decreased during traction by turning the springs 3 in a clockwise or counter clockwise direction, respectively.

In the cadaver model, the dynamic fixator of the invention did not block visualization of the fracture site on a true lateral view, and could maintain reduction of an unstable PIP joint fracture through full range of motion.

Additionally, the dynamic external fixator of the invention is a modular system which is easy to use and quick to assemble. It is not bulky and does not disturb the neighbor fingers, as can be seen on FIG. 8. The maximum distraction force created was approximately 500 g for each side, in the same range as previously reported forces.

The dynamic external fixator of the invention is an adjustable system, which means the traction force on either side can be modified continuously by turning the springs 3 and a lateral tilting deformity could be corrected if imbalance of the fixator occurs during distraction. Moreover, there is no need to calculate the exact position of the distal K-wire K2 according to the size of a pre-assembled fixator since the distal K-wire K2 is inserted through a sliding groove 14 that extends along the longitudinal axis of the of the rod 1.

The fixator of the invention is an elastic dynamic external fixator which not only allows for motion of the PIP joint but also preserves elasticity of the ligaments and capsule during distraction period. The sliding groove is preferably larger than the K-wires, for example 0.2 mm larger, so that slight rotation of the middle phalanx during motion of PIP joint is possible. Rotation is crucial as supination occurs physiologically with PIP joint flexion. Therefore some inaccuracy is tolerated in attachment means placement.

Furthermore, the rods 1 of the dynamic external fixator of the invention can move independently on the attachment means, for example on the K-wires K1, K2 and/or K3, and thus do not interfere with the wire-bone interface during finger motion. In other words, the attachment means rotate relative to the rods and almost do not rotate relative to the bones. This allows avoiding, or at least reducing, pin tract infection and pin loosening rate that may occur from the motion between the K-wire and the bone.

Among various treatment modalities, dynamic distraction can alleviate adhesion of the tendons and peri-articular tissues and prevent contracture of the ligaments and capsule. However, it is not an exclusive method which means it can also be used as in combination with other methods to achieve improved outcome. An application of the dynamic external fixator of the invention is for example PIP joint fracture-dislocation which can be reduced by closed means or by a limited open (percutaneous) technique but remain dynamically unstable. The dynamic external fixator of the invention may also be useful in conjunction with volar plate arthroplasty, hemi-hamate arthroplasty, ORIF, or percutaneous pinning.

The dynamic external fixator of the invention was described above in its use for reduction of PIP joint fracture-dislocations. It can also be used to treat unstable fracture-dislocations of the DIP joint in a reversed position, or for the treatment of intra-articular IP joint and metacarpo-phalangeal (MP) joint fractures of the thumb.

As the PIP joint has the propensity to quickly become very stiff due to the fibroblastic response to injury in surrounding collagenous structures, the fixator of the invention may also be used in distraction arthrolysis for chronic post-traumatic flexion contracture of the PIP joint.

The dynamic external fixator of the invention was described above in its use on the hand, more particularly on the fingers. The size of the external fixator of the invention can however be adapted for its use on any other part of the human body. Larger fixator according to the invention may for example be used for the distraction of the knee or of the elbow, for example be used in distraction arthrolysis for chronic post-traumatic joint stiffness of elbow joint. In such applications, the attachment means are for example Steinmann wires that are thicker than K-wires, or any other appropriate attachment means.

1. A dynamic external fixator comprising:
a pair of rods made of a radiolucent material, each rod of said pair of rods comprising:
a base;
a stem extending from said base, said stem comprising a sliding groove extending along a longitudinal axis of said stem; and
stopping means between said base and said stem;
a pair of springs, each spring of said pair of springs being arranged on the stem of one of said rods and abutting against said stopping means;
proximal attachment means for attaching the base of each of said rods to a first position;
distal attachment means for attaching the stem of each of said rods to a second position, wherein said distal attachment means is inserted into the spirals of the spring and through the sliding groove of each of said rods, such that turning said spring around said stem allows adjusting a distraction force between said proximal attachment means and said distal attachment means.

2. The dynamic external fixator of claim 1, further comprising retaining means for preventing said spring from unwantedly turning around said stem in a direction reducing said distraction force.

3. The dynamic external fixator of claim 2, wherein said stopping means comprises a shoulder of said base and said retaining means comprises a groove formed in said shoulder.

4. The dynamic external fixator of claim 3, wherein the pitch of said spring is reversed at a proximal end of said spring that abuts against said stopping means.

5. The dynamic external fixator of claim 4, wherein the pitch of said spring is reversed at said proximal end on a distance comprised between one and two turns.

6. The dynamic external fixator of claim 1, wherein said base comprises an opening for receiving said proximal attachment means.

7. The dynamic external fixator of claim 1, wherein said stopping means is a shoulder formed between said base and said stem.

8. The dynamic external fixator of claim 1, wherein said stopping means comprises a spacer made of radiolucent material placed on said stem and abutting against a shoulder of said base.

9. The dynamic external fixator of claim 8, wherein said stem comprises a screw thread matching a screw thread of said spacer, wherein the position of said spacer on said stem is determined by screwing/unscrewing said spacer on said stem.

10. The dynamic external fixator of claim 8, wherein said stopping means comprises a plurality of spacers made of radiolucent material and placed next to each other on said stem.

11. The dynamic external fixator of claim 1, wherein said proximal attachment means and said distal attachment means are K-wires.

12. The dynamic external fixator of claim 1, further comprising reduction attachment means for attaching the stem of each of said rods to a reduction position between said proximal position and said distal position, wherein said reduction attachment means are inserted into the spirals of the spring and through the sliding groove of each of said rods, such that the reduction attachment means can be moved within said sliding groove by turning said spring around said stem.

13. The dynamic external ixator of claim 12, wherein said reduction attachment means is a K-wire.

14. A method for attaching the dynamic external fixator of claim 1, said method comprising:
attaching said proximal attachment means to a first position;
attaching said distal attachment means to a second position;
selecting a pair of rods with appropriate position of the stopping means for creating a window of appropriate size free of any material that may obscure an X-ray image;
placing one spring of said pair of springs on the stem of each rod of said pair of rods:
attaching said proximal attachment means to the base of each rod;
inserting said distal attachment means through the spirals of each of said springs and through the sliding groove of each rod;
adjusting the distraction force between said proximal attachment means and said distal attachment means by turning each spring of said pair of springs.

15. The method of claim 14, further comprising the step of attaching reduction attachment means to a third position between said first and said second position and inserting said reduction attachment means through the spirals of each of said springs and through the sliding groove of each rod.

16. The method of claim 14, wherein the step of selecting a pair of rods with appropriate position of the stopping means comprises selecting a pair of rods with appropriate dimensions of the base along a longitudinal axis of said rod.

17. The method of claim 14, wherein the step of selecting a pair of rods with appropriate position of the stopping means comprises selecting one or more spacers for placement on the stem of each of said pair of rods, in abutment against the base of the corresponding rod.

18. The method of claim 14, wherein the step of selecting a pair of rods with appropriate position of the stopping means comprises adjusting a distance between the base and a spacer of each rod by screwing/unscrewing said spacer on the stem of the corresponding rod.

19. A dynamic external fixator comprising:
a pair of rods made of a radiolucent material, each rod of said pair of rods comprising:
a base;
a stem extending from said base, said stem comprising a sliding groove extending along a longitudinal axis of said stem; and
stopping means between said base and said stem;
a pair of springs, each spring of said pair of springs being arranged on the stem of one of said rods and abutting against said stopping means;
proximal attachment means for attaching the base of each of said rods to a first position;
distal attachment means for attaching the stem of each of said rods to a second position,
wherein a window free of any material that may obscure an X-ray image is formed between the proximal attachment means and the stopping means.
